(19) United States
(12) Patent Application Publication
(10) Pub. No.: US 2023/0102810 A1
(43) Pub. Date: Mar. 30, 2023

(54) THE USE OF UNRIPENED GREEN MANGIFERA INDICA AS A NATURAL PREPARATION TO TREAT SARS-COV-2

(71) Applicant: Applied Bio Research Inc., Windsor (CA)

(72) Inventors: Sabah A A Jassim, Windsor (CA); Hassan El-Cheikh, Windsor (CA); Maryam Jassim, Windsor (CA); Issa El-Cheikh, Windsor (CA)

(21) Appl. No.: 17/887,277

(22) Filed: Aug. 12, 2022

Related U.S. Application Data
(60) Provisional application No. 63/248,281, filed on Sep. 24, 2021.

(51) Int. Cl. A61K 36/22; A61K 36/9068; A61K 36/752; A61K 36/71; A61P 39/06; A61P 31/14; A61K 9/08; A61K 9/00

(52) U.S. Cl. CPC A61K 36/22; A61K 36/9068; A61K 36/752; A61K 36/71; A61P 39/06; A61P 31/14; A61K 9/08; A61K 9/0053; A61K 2236/13; A61K 2236/15; A61K 2236/17; A61K 2236/35; A61K 2236/53

(57) ABSTRACT

The present disclosure provides a method for an effective decoction preparation of unripe green Mangifera indica to treat and prevent anti-SARS-CoV-2. The disclosure extends to uses of such preparation as medicaments and to methods of treating anti-SARS-CoV-2 infections, and tolerating the vaccine side-effects. The disclosure further extends to methods for preventing anti-SARS-CoV-2 infections by coating objects and surfaces with the compositions.

The present invention claims priority to U.S. provisional application 63/248,281 filed on Sep. 24, 2021, presently pending, the contents of which are hereby incorporated by reference.

### FIELD

The present disclosure relates to a composition and method for treatment and prevention of SARS-CoV-2. More specifically, the invention relates to a preparation of Mangifera indica for a composition and method to treat SARS-CoV-2.

### BACKGROUND

In the last two decades, there has been an exponential growth in the field of herbal medicine, and these alternative drugs are gaining popularity both in developing and developed countries because of their natural origins and minimal side effects (Jassim and Naji 2003). Furthermore, fruits and vegetables are rich sources of many different bioactive phytocompounds, including phenolic components, which exhibit good antiviral properties and are, therefore, regarded as an unquestionable component that should be the future of phyto-antiviral agents (Jassim and Naji 2003; Jassim 2005). Many traditional medicines in use are derived from medicinal plants, fruits, and vegetables, and in this regard, the World Health Organization (WHO) has listed 21,000 plants used for medicinal purposes around the world. Currently, nearly 30% or more of the modern pharmacological drugs are derived directly or indirectly from plants (Jassim and Naji 2003; Parvez 2016).

Fruit and vegetables are rich sources of many different bioactive phytocompounds, including phenolic components, anthocyanins, carotenoids, vitamin E, and vitamin C, which exhibit good antioxidant properties and are, therefore, regarded as essential components that should be present in everyone's diet (Liu 2003). It has been well documented that mango fruits are an important source of micronutrients, vitamins, and other phytochemicals. Moreover, mango fruits provide energy, dietary fiber, carbohydrates, proteins, fats, and phenolic compounds (Tharanathan et al. 2006), vital to normal human growth, development, and health (Jahurul et al. 2015).

Mangifera indica L. is the species of mango in the Anacardiaceae family (Lauricella et al. 2017). Mango is a rich source of polyphenols, a diverse group of organic micronutrients found in plants that exert specific health benefits (Lauricella et al. 2017). Polyphenols identified in mango mesocarp include mangiferin, gallic acid, gallotannins, quercetin, isoquercetin, ellagic acid, and β-glucogallin (Lauricella et al. 2017), with gallic acid being the most represented phenol compound in this fraction (Lauricella et al. 2017). Furthermore, up to 25 diverse carotenoids have been identified in the mesocarp fraction, such as provitamin A, lutein, α-carotene, and β-carotene that account for the yellowish color of this part of the fruit (Lauricella et al. 2017). Mangiferin, being a polyphenolic antioxidant and aglucosyl xanthone, has strong antioxidant, anti-lipid peroxidation, immunomodulation, cardiotonic, hypotensive, wound healing, antidegenerative, and antidiabetic activities (De and Pal 2014).

Mangifera indica grows in the tropical and subtropical regions and various parts are commonly used in folk medicine for a wide variety of remedies such as antidiabetic, antioxidant, anti-viral, cardiotonic, hypotensive, anti-inflammatory properties, antibacterial, anti-fungal, anthelmintic, anti-parasitic, anti-tumor, anti-HIV, anti-bone resorption, antispasmodic, antipyretic, antidiarrheal, antiallergic, immunomodulation, hypolipidemic, anti-microbial, hepatoprotective, and is gastroprotective. Dentifrice, antiseptic, astringent, diaphoretic, stomachic, vermifuge, tonic, laxative, and diuretic and to treat diarrhea, dysentery, anemia, asthma, bronchitis, cough, hypertension, insomnia, rheumatism, toothache, leucorrhoea, hemorrhage, piles, hair problem, debility, bile related problems, epilepsy, and sunstroke have also been studied (Shah et al. 2010; Juyal and Ghildiyal 2013; De and Pal A. 2014). Also, it was considered that the mangiferin is an antiviral agent for the herpes simplex virus (HSV-1 and HSV-2), HIV, and hepatitis B virus (Parvez 2016). In general, mangiferin, is a molecule primarily obtained from the king of fruits, “Mangifera indica”, and is considered a compound of interest because of its single-molecule therapy approach tested by many researchers (Jyotshna et al. 2016).

### SARS-CoV-2

An outbreak of coronavirus disease 2019 (COVID-19) caused by the 2019 novel severe acute respiratory syndrome coronavirus-2 (SARS-CoV-2) began in Wuhan, Hubei Province, China, in December 2019, and has spread throughout China and to nearly all countries and territories in the world (https://www.ecdc.europa.eu/en/geographical-distribution-2019-ncov-cases).

### Is SARS-CoV-2 Comparable to SARS or to the Seasonal Flu (Influenza)?

The novel coronavirus, COVID-19, detected in China is genetically closely related to the 2003 SARS virus and appears to have similar characteristics, although there is still limited data available on this virus. While both COVID-19 (SARS-CoV-2) and influenza viruses are transmitted from person to person and may cause similar symptoms (e.g., fever, cough, and shortness of breath), the current outbreak is occurring during a time of year when respiratory illnesses from influenza and other viruses, including other coronaviruses that cause the “common cold,” are highly prevalent. Nevertheless, the two viruses are very different and consequently do not behave in the same way. It is still very early to draw conclusions on how SARS-CoV-2 spreads, but preliminary information indicates that SARS-CoV-2 is transmissible in the same way as SARS and some other pandemic influenza strains that have been reported.

### SUMMARY

In the present disclosure, a use and a method for the treatment of SARS-CoV-2 are provided.

Thus, by one broad aspect of the present invention, a use of Mangifera indica in the manufacture of a medicament to treat or prevent one or more of SARS-CoV-2, viral gastroenteritis, or viral pharyngitis is provided.

By a further broad aspect of the present invention, a method of preparing Mangifera indica for treating or preventing SARS-CoV-2, viral gastroenteritis, or viral pharyngitis is provided, the method including separating the Mangifera indica peel, pulp, and seed kernels, cutting the peel, pulp and seed into small pieces, baking the pieces to dry and grinding the dried peel, pulp and seed into a fine powder.

By a further broad aspect of the present invention, a method for preventing viral infections is provided, the method including applying a Mangifera indica extract to an object or surface.

A further understanding of the functional and advantageous aspects of the invention can be realized by reference to the following detailed description and drawings.

### BRIEF DESCRIPTION OF THE DRAWINGS

Embodiments disclosed herein will be more fully understood from the following detailed description taken in connection with the accompanying drawings, which form a part of this application, and in which:

FIG. 1 is a table of results from an embodiment of the present disclosure.

FIG. 2 is a table of results from a further embodiment of the present disclosure.

FIG. 3 is a scatter plot showing results from a further embodiment of the present disclosure.

### DETAILED DESCRIPTION

The following description and figures are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As described herein, Mangifera indica is used in the manufacture of a medicament to treat or prevent one or more of SARS-CoV-2, viral gastroenteritis or viral pharyngitis. The peel, pulp, seed, or a mixture thereof from the Mangifera indica may be used in the manufacture of the medicament.

A method for preparing Mangifera indica for treating or preventing SARS-CoV-2, viral gastroenteritis, or viral pharyngitis is to separate the peel, pulp, and seed kernels of the green fruit, cut the peel, pulp and seed into small pieces, bake the pieces to dry, and grind the dried peel, pulp and seed into a fine powder.

The fine powder may then be further prepared by extracting with an organic solvent, preferably acetone, centrifuging to remove particulate matter, collecting the supernatant, and drying the supernatant in a desiccator.

Alternatively, the fine powder may be further prepared by extracting the fine powder with boiling water and filtering the extract to collect the filtrate.

A method for preventing viral infections includes applying a Mangifera indica extract to an object or surface.

Example embodiments for the preparation and use of fruit (Mangifera indica) powder for the treatment or prevention of viral infections are presented below.

### Plant Material

Unripe green Mangifera indica were purchased from a local market.

### Preparation of Fruit Powder

The fruits were cleaned with water. The mangos were manually separated into the peel, pulp, and seed kernels, cut into small pieces, and were separately placed in an oven at 60° C. for 24 h to dry. After drying each of the peels, pulps, and seed kernels, they are then ground into a fine powder individually. The resulting green slightly fibrous (peel), beige slightly fibrous (pulps), and slightly beige with high fiber (seed kernels) powders were collected and stored in an airtight container for subsequent use.

### Example 1 Chemical Extract of Mango

One (1) gram of powder from the mango's peel, pulp and seed kernels were extracted individually with 30 ml of 70% (v/v) acetone at room temperature. After 72 h, the extracts were then evaporated in an air dryer cabinet at 40° C. After 24 hours, approximately 1 ml left from each extract was collected individually in sterile Eppendorf micro-centrifuge tubes (polypropylene; 1.5 ml; Sarstedt) and centrifuged at 15,000 rpm for 5 min, and the supernatants were collected and stored for 24 h in a desiccator at 8° C. It was noticed that there was a milky precipitate in the mango peel extract and to less extent with the seed kernel extract. No precipitate was found with pulps extract solution.

### Virucidal Activity

Ten microliters of a 1×10⁹ PFU ml⁻¹ Pseudomonas phage ATCC 14209-B1 were placed in sterile 96-well polystyrene plates, then 90 μls were added of each 10, 1, 0.1, 0.01, and 0.001% of the mango extract preparation, and 10% of a mixed cocktail of equal volumes of each extract of peel, pulp, seed kernels. The suspensions were mixed thoroughly. After 15 min of incubation at room temperature, the number of surviving phages was evaluated by 10-fold serial dilution in l-buffer, and 10 μl of each dilution was placed on Pseudomonas aeruginosa ATCC 25668 lawns. The plates were incubated for 18 h at 37° C.

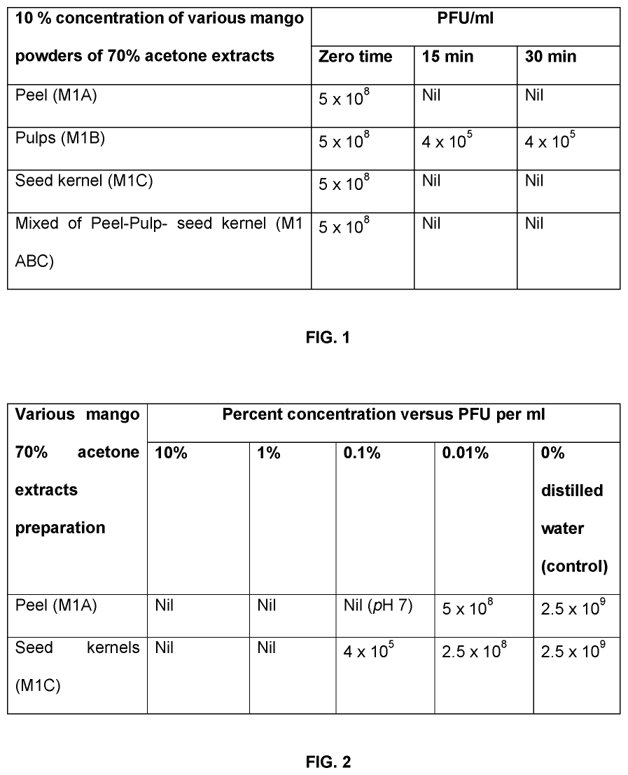

Referring to FIG. 1, antiviral activity (pfu/ml) of various concentrations of mango acetone extract preparations from peel, pulp, and seed kernels on Pseudomonas phage ATCC 14209-B1 are provided. In FIG. 1, the following codes designate:

### M1A: Peel Powder

### M1B: Pulp Powder

M1C: Seed kernel Powder
AC: 70% acetone extraction

Referring to FIG. 2, virucidal activity (pfu/ml) of various concentrations of acetone extract of peel and seed kernels on Pseudomonas phage ATCC 14209-B1 measured after 10 min of contact are provided.

### Results

The unripe green mango peel acetone extract showed stronger antiviral activity than the mango seed kernel acetone extract, in which 0.1% (1 mg ml⁻¹) of the unripe green mango peel acetone extract demolished the pfu to nil, in comparison to the same percentage of the seed kernel extract where pfu was reduced by 4 log cycle. The pH of 0.1% peel extract was 7. The mango peel extract was chosen for further studies of antiviral activities.

### Example 2. Decoction Preparation of Peel Mango Extract

50 g of the mango peel powdered sample was added to 500 ml of distilled water in a 1 liter round bottom flask, and was brought to a boil and stirred with a glass rod continuously for 5 min. It was then allowed to stand for 5 min at room temperature. The supernatant was poured into a clean flask and was then filtered using Whatman filter paper No. 91 (18.5 cm). The filtrate was collected and stored in an airtight container for subsequent use.

In vitro Antiviral Activity

Ten microliters of a 1×10⁹ PFU ml¹ Pseudomonas phage ATCC 14209-B1 were placed in sterile 96-well polystyrene plates, and then 90 μls of 0.1% of peel mango extract was added. The suspensions were mixed thoroughly. After 15 min of incubation at room temperature, the number of surviving phages was evaluated by a 10-fold serial dilution in I-buffer, and 10 μl of each dilution was placed on Pseudomonas aeruginosa ATCC 25668 lawns. The plates were incubated for 18 h at 37° C.

### Results

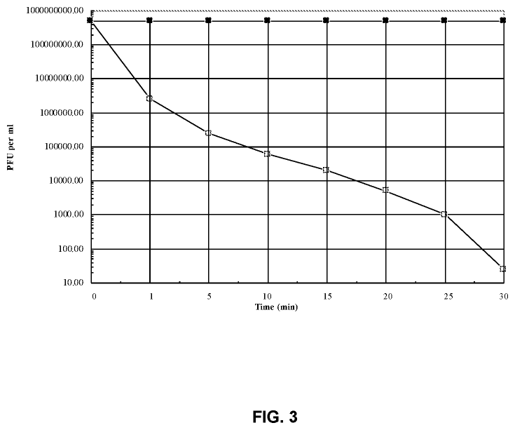

As illustrated in FIG. 3, the virucidal activity with various contact times of 0.1% peel mango powder extract is provided in a scatter plot. Black squares designate distilled water (control sample), and open squares designate 0.1% peel mango powder extract. The D-value was collocated to 7 min. The results demonstrate the virucidal efficacy of 0.1% v/v of unripened green peel mango decoction reduced the bacteriophage ATCC 14209-B1 titer by 2 and 4 log₁₀ cycles after 1 and 10 min, respectively. After 30 min the phage titer was reduced 7 log₁₀ cycles. The D-values were highest at 7 min.

### Novel Herbal Preparation Treatment

### Preclinical Trial Studies

In the following trials, 1% of the peel of unripe green mango decoction extract was used.

### Case Sheet Observations

The following is a sample from over a hundred cases that were observed.

### Viral Gastroenteritis (Stomach Flu)

Influenza viruses are spread by coughing and sneezing. Norovirus and rotavirus are common causes of viral gastroenteritis and are transmitted by the fecal-oral route, passed by contact, and entering the body in food or water.

No. of patients: four patients.

Past medical history: nothing significant which could be related to the current case.

Causative agent: Rotavirus.

### Course of the Infection in General for the 4 Patients:

Semi-abrupt onset of signs and symptoms of gastroenteritis like nausea, vomiting, abdominal cramps, gases, severe diarrhea (semi-watery), and mild-moderate fever with frequent chills, no rigor.

The first and third patients showed signs and symptoms mentioned above for 24 hrs, then the first dose of 100 ml of the unripe green peel mango decoction solutions (1% v/v) was taken orally (crude solution which contains very diluted active substances). One hour later, diarrheal symptoms recurred mildly. Afterward, within 4 hrs, there was an alleviation of symptoms. A second dose (see above) was taken at this time orally. A complete cure was observed within one hour from the second oral dose. A cure was assessed by the demise of fever, chills, diarrhea, vomiting, abdominal cramps, etc.

The second and fourth patients showed the same signs and symptoms as the first and third patients. A first oral dose was taken 12 hrs after the onset of symptoms. Signs and symptoms disappeared completely, and a cure was established within 2 hrs. No second dose was needed.

### Viral Pharyngitis:

Pharyngitis is inflammation of the back of the throat, known as the pharynx. It typically results in a sore throat and fever. Other symptoms may include a runny nose, cough, headache, difficulty swallowing, swollen lymph nodes, and a hoarse voice.

No. of patients: ten patients.

Past medical history: All patients have recurrent viral pharyngitis that frequently occurs every 3 months and sometimes proceeds to a complete case of upper respiratory flu illness.

Causative agent: not known, but highly indicative of viral etiology according to the history and clinical examination.

### Course of the Infection in General for the 10 Patients:

Signs and symptoms were malaise, severe soreness of the throat, mild fever, fatigue, and headache.

All patients showed a gradual occurrence of a sore throat, progressing to severe viral pharyngitis. The first oral dose 100 ml of the unripe green peel mango decoction solutions (1% v/v) was taken 4 hrs from the onset of the disease for the 10 patients. Observable alleviation of symptoms was noticed within 3-4 hrs. The second oral dose was taken after 8 hrs, and a complete cure was observed within 2-3 hrs from the second oral dose.

### SARS-CoV-2:

### Example

One patient (32 years, male) contracted SARS-CoV-2 (COVID-19) after getting vaccinated with 2 doses of the Chinese vaccine. The patient lost the sense of smell and taste, had a high fever, was short of breath, etc. He did not take any medication except an oral dose of 100 ml of the unripe green peel mango decoction solution (1% v/v), and after 24 hours, all signs and symptoms disappeared completely, and a cure was established. No second dose was needed.

A family of 5 persons contracted COVID-19 (father 59-year-old, mother 48-year-old, children aged 21, 19, and 17 years). The patients lost their sense of smell and taste, had a high fever, were short of breath, etc. They did not take any medication except an oral dose of 100 ml of the unripe green peel mango decoction solution (1% v/v) 3 times a day for 3 days, and after 24-72 hours, all signs and symptoms disappeared completely, and a cure was established.

One patient (21 years female) contracted SARS-CoV-2 (COVID-19) after 2 days of being vaccinated with 2 doses of the Pfizer vaccine. The patient lost her sense of smell and taste, had a high fever, severe headache, shortness of breath, etc., and was diagnosed positive with SARS-CoV-2. She did not take any medication except an oral dose of 100 ml of the unripe green peel mango decoction solution (1% v/v) 3 times a day, for 7 days, and after 48-72 hours all signs and symptoms disappeared completely, and the complete recovery was established after 5 days. The patient tested negative for SARS-CoV-2 on day 7. It was noticed that her family (mother 46 y, sisters 10 y and 17y, and 2 brothers 13 y and 19 y), all vaccinated with a first dose of Pfizer, and living with her at the same house, also took an oral dose of 100 ml of the unripe green peel mango decoction solution (1% v/v) once a day and they did not contract SARS-CoV-2. After taking the decoction solution once a day for 7 days they all tested negative for SARS-CoV-2, showing that the decoction solution can also prevent SARS-CoV-2 infection. This result has been observed in many cases.

A patient (76 years, male, heavy smoker) contracted SARS-CoV-2 (COVID-19). The patient lost his sense of smell and taste, had a high fever, severe headache, shortness of breath, pain in the chest, etc. He tested positive for SARS-CoV-2. He took Tylenol to help reduce his fever but did not take any other medication with the Tylenol except an oral dose of 100 ml of the unripe green mango peel decoction solution (1% v/v) for 14 days. After day 10, most of his signs and symptoms disappeared, and complete recovery was established after day 14, in which he tested negative for SARS-CoV-2 on day 15.

The Mangifera indica extract is also effective on people receiving a COVID-19 vaccine. Such people usually get headaches and chills and develop a fever for one and a half days following vaccination. The Mangifera indica extract is beneficial for tolerating the vaccine side effects and strengthening people to overcome side effects following vaccination.

A plant extract with antiviral activity, preferably Anti-SARS-CoV-2 activity, is an extract of unripe Mangifera indica, and preferably a peel and seed kernels extract. This extract exhibits the surprising activity of treating and preventing SARS-CoV-2.

Compositions according to the invention comprise an extract of the peel and seed kernels from unripe green mango spp. The inventors have found that extracts from the whole unripe green mango may be effectively used to provide anti-SARS-CoV-2 compositions according to the invention. However, preferably compositions, according to the invention, comprise an extract from the unripe green mango fruit, i.e. preferably the green unripe mango's peel and seed kernel extract.

The unripe green mango peel and seed kernel extract have been shown to have an active component. Preferably, compositions according to the invention comprise a peel, pulps, and seed kernel extract of unripe green mango and in particular unripe green Mangifera indica. Any parts of the unripe green mango may be used to prepare compositions according to the invention, although preferred teas are a tea of the mixed peel, pulps, and seed kernel powders of unripe green mango. As provided in the example above, a combination of powder of green mango tea has an effective prevention effect and acts as a treatment for patients that have contracted SARS-CoV-2.

In order to prepare suitable plant extracts for preparing compositions according to the invention, the chosen plant is first comminuted, preferably in a solvent, which is then preferably boiled. A preferred solvent is water. The extract may be fractionated, preferably by centrifugation. The fractionated extracts contain an active compound.

The example above established that the preferred extraction method for green unripe mango was by boiling at about 100° C. for at least 2 min, more preferably for at least 4 min, even more preferably for at least 6 min, and most preferably for at least 10 min. The plant extracts may be sterilized, for example, by autoclaving and then allowed to cool and stored at −20° C. The plant extract may be used in a concentrated form. Alternatively, the extract may be diluted as appropriate to its intended use. Typically, about 10 g of dried plant extract may be used in about 300 ml of water. This may give an effective concentration of between about 1 and 99% (w/w) plant extract, preferably between about 2 and 80% (w/w) plant extract, and more preferably between about 5 and 50% (w/w) plant extract. Effective compositions, according to the invention, comprise 99-1% (v/v) of the unripe green mango extract. The compositions, according to the invention, may be in the form of a solid or liquid concentrate for dilution with water.

Due to their increased biological activity, the compositions of the invention may be used in the treatment of any viral infection. Preferably, the compositions according to the invention are anti-viral compositions. The virus may be a DNA or an RNA virus. For example, viruses against which the compositions in accordance with the invention are effective may include SARS-CoV-2, Flu, viral gastroenteritis (stomach flu), such as Norovirus and rotavirus, viral pharyngitis, and bacteriophage as demonstrated in Example 1. Compositions and medicaments, according to the invention, may be used in the prevention and treatment of several viral infections. The virus may be any virus, and particularly an enveloped virus. Examples of viruses against which the compositions are effective include poxviruses, iridoviruses, togaviruses, or toroviruses. Further examples include a filovirus, arenavirus, bunyavirus, and rhabdovirus. Further examples include a paramyxovirus or an orthomyxovirus. It is envisaged that viruses may be a hepadnavirus, flavivirus, or retrovirus. The virus may be a herpes virus or a lentivirus. The virus may be Human Immunodeficiency Virus (HIV), Human herpes simplex virus type 2 (HSV2), or Human herpes simplex virus type 1 (HSV1).

### Example 3 Formula

The following formula is a further example:
Mangifera indica “Mango” powder 95%. 6.65 g
Zingiber officinale “Ginger” 2% 0.14 g
Citrus bioflavonoids “Citrus limon” 2% 0.14 g
Nigella sativa “Black cumin” 1% 0.07 g

Serving size: 7 g packet

Taken three times per day

The above formula provides antioxidant(s) that help fight and reduce oxidative damage and cell damage caused by free radicals. It further helps prevent nausea and vomiting associated with motion sickness and/or seasickness. The antioxidant provides a benefit to protect cells against and reduce the oxidative effect of (the oxidative damage caused by/cell damage caused by) free radicals. Other benefits include function as an expectorant and cough suppressant (antitussive) to help relieve bronchitis as well as coughs and colds. In addition, the formulations help relieve digestive upset/disturbances, including lack of appetite, nausea, digestive spasms, indigestion, dyspepsia, and flatulent colic (carminative).

It will be appreciated that the compositions according to the invention may be used in monotherapy (i.e. use of the compositions according to the invention alone to prevent and/or treat a viral infection or contamination). Alternatively, the compositions according to the invention may be used as an adjunct to, or in combination with, known anti-viral therapies. For example, compounds used in antiviral therapy include but are not limited to acyclovir, ganciclovir, ribavirin, interferon, anti-HIV medicaments, including nucleoside, nucleotide or non-nucleoside inhibitors of reverse transcriptase, protease inhibitors, and fusion inhibitors. Hence, compositions and medicaments, according to the invention, may be used in combination with such antiviral agents.

Compositions, according to the invention, may have several different forms depending on the way the composition is to be used. Thus, for example, the composition may be in the form of a powder, tablet, capsule, liquid, lozenges, ointment, gel, hydrogel, aerosol, spray, micellar solution, transdermal patch, liposome suspension, or any other suitable form that may be administered to a person or animal. It will be appreciated that the vehicle of the composition of the invention should be one which is well tolerated by the subject to whom it is given.

Compositions and medicaments comprising plant extract according to the invention may be used in several ways. For instance, oral administration may be required in which case the plant extract may be contained within a composition that may, for example, be ingested orally in the form of a tablet, capsule, lozenges, or liquid. The compositions may also be administered by inhalation (e.g., intranasally). Alternatively, compositions according to the invention may be administered by aerosol, for example using an atomiser, by which the composition may be administered nasally or via the lungs.

However, the compositions may be topically applied, for example in the form of an ointment, cream, gel, or aqueous solution. Topical administration is useful when a subject to be treated is suffering from a viral infection. For instance, ointments, for example, CamOleum ointment available from Camoleum, Inc., located in Windsor, Ontario, Canada, applied to the peel, areas in and around the mouth, or genitals to treat specific viral infections. The composition may be applied intravaginally (for example, if required to protect the subject from sexually transmitted diseases), or rectally. Intravaginal administration is effective for treating sexually transmitted diseases (including AIDS). Topical application to the peel is particularly useful for treating viral infections of the peel or as a means of transdermal delivery to other tissues also.

Generally, a daily dose of 100 ml for adults and children (ages 10 years-16 years), according to the invention, may be used for the prevention and/or treatment of a viral infection, depending on which composition is used.

Compositions, according to the invention, can be administered orally in the form of a sterile solution or suspension containing other solutes or suspending agents (for example, enough saline or glucose to make the solution isotonic), honey, bile salts, acacia, gelatin, and the like. Compositions according to the invention can also be administered orally either in liquid or solid composition form. Compositions suitable for oral administration include solid forms, such as pills, capsules, granules, tablets, lozenges, powders, and liquid forms, such as solutions, syrups, elixirs, and suspensions. Forms useful for parenteral administration include sterile solutions, emulsions, and suspensions.

Compositions according to the invention may have the form of solutions, suspensions, emulsions, syrups, elixirs, and pressurized compositions. The active agents may be dissolved or suspended in a pharmaceutically acceptable liquid vehicle such as water, an organic solvent, a mixture of both, or pharmaceutically acceptable oils or fats.

The liquid vehicle may contain other suitable pharmaceutical additives such as solubilizes, emulsifiers, buffers, preservatives, sweeteners, flavoring agents, suspending agents, thickening agents, colors, viscosity regulators, stabilizers, or osmoregulators. Suitable examples of liquid vehicles for oral and parenteral administration include water (containing additives as above, e.g., cellulose derivatives, alcohols (including monohydric alcohols and polyhydric alcohols, e.g., glycols) and their derivatives, and oils (e.g,. fractionated camel oil, coconut oil and arachis oil).

The compositions according to the invention may be used to treat any mammal, for example, human, livestock, pets, and may be used in other veterinary applications. The inventor has realized that the compositions, according to the invention, may be used as a medicament but may also be put to several other anti-viral uses (whether in a clinical context or otherwise). For instance, in addition to administering the compositions according to the invention to a patient or subject, they may be used for the application to, or coating of, surfaces and objects to prevent, ameliorate, or treat viral infections or contaminations.

Therefore, according to these aspects, there is provided a method of preventing and/or treating a viral infections or contaminations. The method comprises applying to an object or a surface an amount of a composition that is effective for killing or preventing viral amplification, wherein the composition comprises an extract of a plant selected from a group consisting of (i) unripened green mango e.g., Mangifera indica (ii) an extract of a plant selected from a group consisting of unripe green mangos.

As discussed above, the compositions according to the invention have the advantage that they are antiviral. Accordingly, the compositions disclosed herein have a broad anti-viral effect. Furthermore, as discussed in more detail below, the compositions may adhere to surfaces and are thereby effective for longer periods of time.

The compositions according to the invention may be used for application to, or coating of, any object or device which is used in a biological or medical situation, such as a medical device, and for which it may be important to prevent a viral infection or contamination that may lead to any infection in a patient. Examples of medical devices to which compositions according to the invention may be applied include lenses, contact lenses, catheters, stents, wound healing dressings, contraceptives, surgical implants, and replacement joints. The compositions are particularly useful for coating biomaterials and objects and devices made therefrom. Viral contamination/infection of biomaterials can be particularly problematic because the virus may use such material as a substrate for contamination. Biomaterials (e.g., collagens and other biological polymers) may be used to surface artificial joints. Alternatively, certain implants may substantially comprise such biomaterials.

The compositions may be used to coat surfaces in environments that are required to be aseptic. For instance, the compositions may be used in medical environments. The compositions may be used to keep hospital wards clean. They may be used to clean surfaces of medical equipment (e.g., operating tables) in hospitals, such as operating theatres as well as operating theatre walls and floors. The compositions may be useful to improve sterility in general, and to address the spread of SARS-CoV-2. In particular, SARS-CoV-2 may be killed by the compositions of the invention. Therefore, the method according to these aspects may comprise applying the composition to a surface that is selected from: hospital ward surfaces, operating theatre surfaces, kitchen surfaces and sanitary surfaces. It will be appreciated that the above list of objects and surfaces to which compositions according to the invention may be applied is not exhaustive. Hence, the compositions may be administered to any surface, which is prone to a viral contamination, for example kitchen and bathroom surfaces and products, such as a toilet seat, or the toilet itself. The compositions may be formulated into solutions for cleaning objects and surfaces, or for spraying thereon, or in which the object or surface may be immersed. For instance, there may be a routine constituent of physiological solutions (for example as a constituent of physiological saline). Preferably, coating of the object or surface may be carried out by preparing an aqueous solution at an appropriate pH and temperature for the composition, according to the invention, to retain its antiviral activity. The object or surface is exposed to the composition for sufficient time to allow immobilisation or absorption of a suitable quantity of the composition to the surface thereof or to kill the viruses.

Furthermore, the compositions according to the invention may be used to minimize, prevent, or treat viral infections or contaminations, by use as, or in conjunction with, a preservative. Hence, the compositions may be used as a preservative in foodstuffs. In addition, the compositions may be used to minimise or prevent viral growth in cultures, for example in tissue culture work, either to supplement or to replace antiviral. All the features described herein (including any accompanying claims, abstract, etc.), and/or all the steps of any method or process so disclosed, may be combined with any of the above aspects in any combination, except combinations where at least some of such features and/or steps are mutually exclusive.

While the applicant's teachings described herein are in conjunction with various embodiments for illustrative purposes, it is not intended that the applicant's teachings be limited to such embodiments. On the contrary, the applicant's teachings described and illustrated herein encompass various alternatives, modifications, and equivalents, without departing from the embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

### REFERENCES

De P K and Arna Pal A. 2014. Effects of aqueous young leaves extract of Mangifera indica on gm (−) microorganisms causing gastrointestinal disorders. Asian Journal of Plant Science and Research 4(1):23-27
Jahurul M H A, Zaidul I S M, Ghafoor K, et al. 2015. Mango (Mangifera indica L.) by-products and their valuable components: A review. Food Chemistry. 183:173-180.
Jassim S. A. A. 2005. Novel phyto-anti-HIV drugs: A cause for optimism, Biologist (UK) 52 (5), 268-272.
Jassim S. A. A. and Naji M. A. 2003. Novel antiviral agents: A medicinal plant perspective. Journal of Applied Microbiology (UK) 95, 412-427.
Juyal P, Ghildiyal J C 2013. Medicinal phyto-diversity of bhabar tract of garhwal Himalaya. Journal of Medicinal Plants Studies 1(6):43-57.
Jyotshna, Khare P, Shanker K 2016 Mangiferin: A review of sources and interventions for biological activities. Biofactors 42(5):504-514. doi: 10.1002/biof.1308
Lauricella M, Emanuele S, Calvaruso G, et al. 2017 Mangifera indica L. (Mango): The inestimable value of orchards recently planted in Sicilian rural areas. Nutrients 9:525. doi:10.3390/nu9050525
Liu R H. 2003. Health benefits of fruit and vegetables are from additive and synergistic combinations of phytochemicals. Am. J. Clin. Nutr. 78, 517S-520S.
Parvez G M M 2016. Pharmacological activities of Mango (Mangifera indica): A Review. Journal of Pharmacognosy and Phytochemistry 5(3):1-7.
Shah K A, Patel M B, Patel R J, Parmar P K. 2010. Mangifera Indica (Mango). Phcog Rev 4:42-48. https://www.ncbi.nlm.nih.gov/pmc/articles/PMC3249901/.
Tharanathan R N, Yashoda H M, Prabha T N. 2006. Mango. (Mangifera indica L.), the king of fruits—A review. Food Reviews International. 22:95-123.

1. A use of Mangifera indica in manufacture of a medicament to treat or prevent one or more medical conditions.

2. The use of Mangifera indica as in claim 1, wherein said medical conditions comprise one or more of SARS-CoV-2, viral gastroenteritis, or viral pharyngitis.

3. The use of Mangifera indica as in claim 1, wherein the Mangifera indica comprises peel, pulp, seed, or a mixture thereof.

4. The use of Mangifera indica as in claim 1, wherein the medicament comprises a nasal spray.

5. A method of preparing Mangifera indica for treating or preventing SARS-CoV-2, viral gastroenteritis, or viral pharyngitis, the method comprising:
separating peel, pulp, and seed kernels of the Mangifera indica;
cutting the peel, pulp, and seed into small pieces;
baking the pieces to dry; and
grinding the dried peel, pulp, and seed into a fine powder.

6. The method as in claim 5, further comprising:
extracting the fine powder with an organic solvent;
evaporating the organic solvent;
centrifuging the extract to remove particulate matter;
collecting a supernatant; and
drying the supernatant in a desiccator.

7. The method as in claim 6, wherein the organic solvent is acetone.

8. The method as in claim 5, further comprising:
extracting the fine powder with boiling water; and
filtering the extract to collect a filtrate.

9. The use of Mangifera indica as in claim 1, wherein the use is adapted as a method for preventing viral infections, the method comprising:
applying a Mangifera indica extract to an object or surface.

10. The use of Mangifera indica as in claim 1, where the use is adapted as a method for treating and preventing viral infections, the method comprising:
providing a Mangifera indica extract in a tablet, capsule, liquid, lozenge, cream, ointment, gel, hydrogel, aerosol, spray, micellar solution, transdermal patch, liposome suspension, or any other suitable form that may be administered to a person or animal.

11. The use of Mangifera indica as in claim 1, wherein the use is adapted as a method for treating and preventing viral infections, the method comprising:
applying a Mangifera indica extract to a viral peel, areas in and around a mouth, or to genitals.

12. A method of preparing Mangifera indica for treating people receiving a COVID-19 vaccine to tolerate the vaccine side-effects and to strengthen people against the vaccine side-effects, the method comprising:
separating peel, pulp and seed kernels of the Mangifera indica;
cutting the peel, pulp and seed into small pieces;
baking the pieces to dry; and
grinding the dried peel, pulp and seed into a fine powder.

13. The method of claim 12, further comprising:
extracting the fine powder with an organic solvent;
evaporating the organic solvent;
centrifuging the extract to remove particulate matter;
collecting a supernatant; and
drying the supernatant in a desiccator.

14. The method of claim 12, further comprising:
extracting the fine powder with boiling water; and
filtering the extract to collect a filtrate.

15. The method of claim 5, further comprising incorporating additives to the fine powder of Mangifera indica.

16. The method of claim 15, wherein the additives comprise five percent of total composition, and the additives comprise:
two precent of Zingiber officinale; two percent of Citrus bioflavonoids; and one percent of Nigella sativa.

17. The method of claim 16, wherein resulting composition is taken three times a day.

18. The method of claim 16, wherein each serving size comprises 7 grams.

19. The method of claim 16, wherein the composition is adapted to protect cells against oxidative effect of free radicals.
